(19) United States
(12) Patent Application Publication
(10) Pub. No.: US 2004/0146464 A1
(43) Pub. Date: Jul. 29, 2004

(54) Compositions and method for daily tooth and gum care

(76) Inventor: Joel E. Bernstein, Deerfield, IL (US)

Correspondence Address: JONES DAY, 77 WEST WACKER, CHICAGO, IL, 60601-1692, US

(21) Appl. No.: 10/349,603

(22) Filed: Jan. 23, 2003

(51) Int. Cl. A61K 7/16

(52) U.S. Cl. 424/49

(57) ABSTRACT

A toothpaste composition for providing the benefits of reducing inflammation of the gums, as well as sensitivity of the teeth to extremes of temperature, comprises about 0.001% to 0.1% by weight of capsaicin in a toothpaste vehicle.

### BACKGROUND OF THE INVENTION

Toothpastes have long been used for the daily cleansing of both teeth and gums. Such pastes are generally packaged in tubes and applied to the teeth and gums through means of a small brush termed the “toothbrush.” While many toothpastes are traditional pastes, i.e. powdery materials suspended in a lotion base, some toothpastes of more recent vintage are cream or gel emulsions. Apart from the detergent cleansers included in toothpastes, several other chemicals and ingredients have been included in toothpastes to either reduce the formulation of caries (e.g. fluoride) or to enhance the cleansing (e.g. sodium bicarbonate) or whitening properties of the paste (e.g. carbamide peroxide). Capsaicin (trans-8-methyl-N-vernally-6-nonenamide) is a natural substance extracted from fruits of plants of the nightshade family which has found its principal use as an external analgesic when applied topically to the skin. Capsaicin and less pure forms of the substance, termed capsicum oleoresin or capsaicin oleoresin, are also added in extremely small amounts to commercially prepared foods on account of their pungency properties. Since small amounts of capsaicin are present in the diets of most people in the world, capsaicin has been classified as Generally Recognized As Safe (GRAS) for inclusion in food substances.

Medicinally, capsaicin has been employed as an external analgesic agent in vehicles for application to the skin for relief of arthritis and neuropathic pain. I am the inventor of record on several patents employing capsaicin in such a manner (U.S. Pat. Nos. 4,486,450, 4,536,404 and 5,063,060). However, heretofore the use of capsaicin-containing topical analgesic formulations on mucous membranes (e.g. lips, gums, cheeks) has been contraindicated as it has been thought to be harmful to such tissues and without therapeutic value to conditions involving such tissues. Consequently, all external analgesic products containing capsaicin contain the U.S. Food & Drug Administration mandated warning to avoid contact with mucous membranes.

I have discovered, surprisingly, that capsaicin applied to the gums of the mouth may produce beneficial rather than harmful actions. In particular, I have discovered that capsaicin can be incorporated into toothpaste vehicles, and that when such capsaicin containing toothpastes are used for daily oral care, such capsaicin toothpastes can provide the unexpected benefits of reducing inflammation of the gums and/or decreasing sensitivity of the teeth to temperature extremes from hot or cold beverages.

### DETAILED DESCRIPTION OF THE PREFERRED EMBODIMENTS

I have investigated the possible beneficial effects of applying the naturally occurring substance capsaicin to the gums by means of incorporating capsaicin into toothpastes for daily use by individuals who may suffer from, or be at high risk of suffering from painful and/or inflammatory diseases of the gums or teeth. Individuals utilizing such capsaicin-containing toothpastes for daily cleansing of teeth and gums noted less pain, redness, and swelling of inflamed gums, as well as decreased sensitivity of the teeth and gums to temperature extremes.

In the practice of the invention, concentrations of capsaicin varying from about 0.001% by weight to about 0.1% by weight will be incorporated into toothpastes, of the classical paste variety or of the cream or gel emulsion variety, and such toothpastes will be employed by individuals for daily oral hygiene as they would employ any other toothpaste. Such toothpastes may also contain other agents for other purposes (e.g. anti-cavity, whitening) such as fluorides, sodium bicarbonate or carbamide peroxide. The preferred amount of capsaicin will be from 0.001% to 0.025% by weight of the carrier toothpaste.

The following examples further illustrate the invention. In these examples, all percentages are by weight of the carrier.

### EXAMPLE 1

A fluoride containing toothpaste containing 0.01% capsaicin was utilized to brush the teeth and gums twice daily for 3 months by a 59-year old male with pain and swelling of some areas of the gum, as well as excruciating pain in those areas of the gum upon ingesting very hot or very cold beverages. At the end of the 3-month period, the gums were no longer swollen or painful and the individual could drink hot or cold beverages without noticeable discomfort.

### EXAMPLE 2

A fluoride and sodium bicarbonate toothpaste containing 0.005% capsaicin was utilized for once daily brushing by a 58-year-old female with red, painful gums for 6 months. At the end of the 6 months, her gums were no longer red or painful.

### EXAMPLE 3

A 32 year old male with exquisite sensitivity to temperature extremes of food and beverages manifested by acute sharp tooth pain brushed twice daily with a toothpaste containing 0.025% capsaicin for one month. At the end of the month, he no longer experienced tooth pain on eating hot or cold foods.

It will be apparent to those skilled in the art that only several preferred embodiments have been described by way of example, and that there are various modifications that fall within the scope of this invention.

What is claimed is:

1. A toothpaste composition for providing the benefits of reducing inflammation of the gums, as well as sensitivity of the teeth to extremes of temperatures, said compositions comprising capsaicin (trans-8-methyl-N-vanillyl-6-nonenamide) in an amount of about 0.001% to 0.1% by weight and a pharmaceutically acceptable toothpaste vehicle, said composition suitable for use in daily oral care of the teeth and gums.

2. The composition of claim 1 wherein said toothpaste vehicle is a paste, a cream or a gel.

3. The composition of claim 1 or 2 wherein the composition further comprises one or more other active ingredients selected from the group consisting of anti-cavity ingredients, cleansers, or whiteners.

4. The composition of claims 1, 2 or 3 wherein capsaicin is present in the amount of 0.001% to 0.025% by weight.

5. A method of treating inflammation of the gums or temperature sensitivity of the teeth comprising brushing the teeth and gums with a toothpaste containing capsaicin in the amount of 0.001% to 0.1%.

6. The method of claim 5 wherein said capsaicin is present in the amount of about 0.001% to 0.025% by weight.

7. The method of claim 5 wherein the composition further comprises one or more other active ingredients selected from the group consisting of anti-cavity ingredients, cleansers and whiteners.
